(19) United States
(12) Patent Application Publication
(10) Pub. No.: US 2010/0098770 A1
(43) Pub. Date: Apr. 22, 2010

(54) SIROLIMUS PHARMACEUTICAL FORMULATIONS

(76) Inventors: Manikandan Ramalingam, Kanchipuram (IN); Raviraj Sukumar Pillai, Hyderabad (IN); Kumaran Venugopal, Chennai (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/578,776

(22) Filed: Oct. 14, 2009

Related U.S. Application Data
(60) Provisional application No. 61/141,466, filed on Dec. 30, 2008.

(30) Foreign Application Priority Data
Oct. 16, 2008 (IN) 2548/CHE/2008

(51) Int. Cl. A61K 9/50; A61K 31/4355; A61K 38/13; A61K 31/56; A61P 37/06

(52) U.S. Cl. 424/493; 514/291; 514/11; 514/171

(57) ABSTRACT

Pharmaceutical formulations comprising sirolimus or its derivatives, processes for preparing formulations comprising sirolimus, and their methods of use, treatment, and administration.

Aspects of the present invention relate to pharmaceutical formulations comprising sirolimus or its derivatives. Further aspects of the invention relate to processes for preparing formulations comprising sirolimus and their methods of use, treatment, and administration.

The drug compound having the adopted name “sirolimus” is a macrocyclic lactone produced by Streptomyces hygroscopicus. A chemical name for sirolimus (also known as “rapamycin”) is (3S,6R,7E,9R,10R,12R,14S,15E,17E,19E,21S,23S,26R,27R,34aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]oxaazacyclohentriacontine-1,5,11,28,2 (4H,6H,31H)-pentone. It has a molecular formula C₅₁H₇₉NO₁₃, molecular weight of 914.2, and structural Formula I.

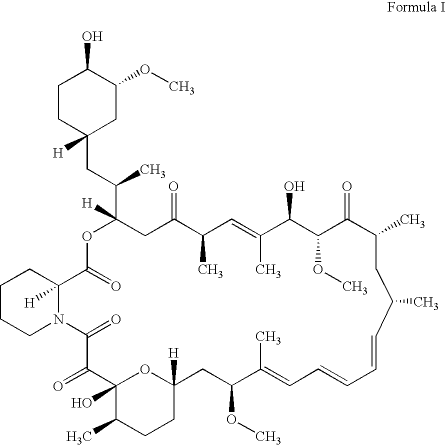

A commercially available formulation containing sirolimus is sold by Wyeth, using the trademark RAPAMUNE®. RAPAMUNE is available in the form of 1 mg and 2 mg oral tablets. RAPAMUNE is indicated for the prophylaxis of organ rejection in renal transplantation.

Rapamycin, its preparation, and its antibiotic activity were described in U.S. Pat. No. 3,929,992.

U.S. Pat. Nos. 5,516,770, 5,530,006, 5,536,729, 5,559,121, 5,145,684, 5,989,591, and 5,985,325, U.S. Patent Application Publication Nos. 2003/0054042 and 2008/0138405, and International Application Publication No. WO 2007/091059 disclose compositions and/or formulations of sirolimus.

U.S. Pat. Nos. 5,989,591 and 5,985,325 disclose a sirolimus solid dosage unit which comprises a core and a sugar overcoat, wherein the sugar overcoat comprises the sirolimus, a surface modifying agent, and a sugar, which coat a core. Optionally, if one or more binders are included in the formulation, they are also considered part of the sugar overcoat. The '591 patent also discloses a process for preparing a sirolimus oral dosage tablet which comprises preparing sirolimus dispersion in poloxamer 188, adding sugars and binders, spraying the dispersion onto a core, and drying.

The commercially available sirolimus formulation appears to be prepared by the process of sugar coating onto a core. The sugar overcoat appears to have sucrose the range of 35-99% by weight of sugar overcoat. Although sugar-coating was popular in the past, the process suffers from many disadvantages such as inability to be applied as a thin film coating, high risk of cracking, the tedious and time-consuming nature of the process, and a requirement for expertise of a highly skilled technician. The poor water solubility of sirolimus poses an issue in formulating the drug into a suitable dosage form. In addition, it has been reported that formulations of sirolimus with conventional excipients can show unpredictable dissolution rates, irregular bioavailability profiles, and instability problems. These formulations further may be susceptible to degradation.

There is a need for formulations that are stable and bioavailable, and are simple and easy to manufacture so that they are easily processed and do not require the expertise of a highly skilled technician.

### SUMMARY

Aspects of the present invention relate to pharmaceutical formulations comprising sirolimus or its derivatives. Further aspects of the invention relate to processes for preparing formulations comprising sirolimus and their methods of use, treatment, and administration.

In embodiments, the invention includes pharmaceutical formulations comprising sirolimus, wherein the formulations may be in monolithic form, or multi-particulate form, or a combination of both.

In an embodiment the invention includes pharmaceutical formulations comprising sirolimus wherein the formulations, which are monolithic or multi-particulate, may be in matrix and/or reservoir forms.

In an aspect the invention relates to pharmaceutical formulations comprising sirolimus, wherein an embodiment comprises:

a) a inert core;

b) a drug layer comprising sirolimus and at least one surface modifying agent surrounding the core;

c) optionally, a barrier layer over the drug coated core; and

d) a sugar coating.

In embodiments, the invention includes pharmaceutical formulations of sirolimus comprising an inert core and a sugar overcoat, wherein the said sugar overcoat comprises: (a) sirolimus or derivatives thereof; (b) at least one surface modifying agent; (c) one or more sugars in an amount of less than about 35% of the weight of the sugar overcoat.

In embodiments, the invention includes pharmaceutical formulations of sirolimus comprising: (a) an inert core; (b) a sugar overcoat comprising: (i) sirolimus or derivatives thereof; (ii) at least one surface modifying agent; and (iii) one or more sugars in an amount less than about 35% of the weight of the sugar overcoat layer; (c) optionally, an inert barrier layer; and (d) a smooth coating layer comprising at least mannitol, and any additional layer thereof.

In an aspect the invention relates to pharmaceutical formulations comprising sirolimus, wherein an embodiment comprises:

a) a inert core;

b) a drug layer comprising sirolimus, at least one surface modifying agent, and a sugar, surrounding the core, wherein the sugar is present in an amount less than about 35% of the weight of the drug layer;

c) optionally, an inert barrier layer applied onto the drug coated core; and

d) a sugar coating.

### BRIEF DESCRIPTION OF THE DRAWINGS

FIG. 1 shows comparative X-ray powder diffraction (“XRD”) patterns of crystalline sirolimus (A), a composition prepared according to Example 8, but without sirolimus (B), a composition prepared according to Example 8 (C), a composition of Example 8 after storage at 40° C. and 75% relative humidity (“RH”) for one month (D), and a composition of Example 8 after storage at 40° C. and 75% RH for two months (E).

FIG. 2 shows comparative XRD patterns of crystalline sirolimus (A), a composition prepared according to Example 9, but without sirolimus (B), and a composition prepared according to Example 9 (C).

### DETAILED DESCRIPTION

Aspects of the present invention relate to pharmaceutical formulations comprising sirolimus, including its pharmaceutically acceptable derivatives. Additional aspects of the invention relate to pharmaceutical formulations comprising compositions of sirolimus or its derivatives. Further aspects of the invention relate to processes for preparing formulations comprising sirolimus, and their methods of use, treatment, and administration.

The “pharmaceutically acceptable derivatives” of sirolimus include, but are not limited to, salts, polymorphs, solvates, esters, hydrates, enantiomers, racemates, and mixtures thereof.

The term “solubility enhanced form” in the context of the present invention refers to sirolimus, or a sirolimus powder composition, that has a higher solubility and/or dissolution rate, as compared to sirolimus in its crystalline form. Such improved solubility properties of sirolimus can be obtained by use of emulsifiers, solubilizers, coprecipitates or solid dispersions, premixes, inclusion complexes, use of amorphous or alternate crystalline forms, and the like, including combinations thereof.

The term “solubility properties” as used herein refers to either an improvement in the solubility of sirolimus, or a modification in the rate of dissolution or a modified absorption of sirolimus.

In embodiments, the formulations of the present invention comprise powder compositions containing sirolimus of derivatives thereof.

“Powder compositions” as used herein refers to either sirolimus powders of defined physicochemical characteristics, or a composition comprising sirolimus together with other excipients in the forms of coprecipitates, premixes, solid dispersions, admixtures with surfactants and/or cyclodextrins, particles of a defined particle size together with emulsifiers and wetting agents, and the like.

The term “premix” or “coprecipitate” or “solid dispersion” as used herein refers to powder compositions comprising sirolimus in intimate or non-intimate mixture with one or more pharmaceutically acceptable polymers.

The term “pharmaceutical acceptable polymer” as used in the context of the present invention refers to any polymers and/or copolymers that enhance the solubility and/or stability of sirolimus.

The term “inert core” as used herein comprises a pharmacologically inactive tablet, core, or inert beads or spheres which comprise one or more of soluble or insoluble inert materials and the like, or mixtures thereof. The cores may be optionally seal coated to increase the strength of the core to withstand the mechanical pressures during processing.

The term “sugar overcoat” as used herein comprises a layer which coats the core. It comprises sirolimus, surface modifying agent, a sugar, and one or more pharmaceutically acceptable excipients. The coating can exist as one or more layers, optionally having one or more separating functional or non-functional layers sandwiched between.

The terms “surface modifying agent,” “surfactant,” and “wetting agent” are synonymous and as used herein include agents which are used to disperse the drug in a particular solvent and also enhance wetting properties of the drug.

The term “pharmaceutical formulation” as used herein refers to a solid dosage form suitable for administration, such as a tablet, capsule, granules, pill, etc.

The term “stability” as used in the invention refers to physical stability and chemical stability, wherein physical stability refers to retaining an original form in the composition and chemical stability refers to resistance to drug degradation and/or impurity generation.

In embodiments, the invention includes pharmaceutical formulations comprising sirolimus, wherein the formulations may be in a monolithic form, multiparticulate form, or a combination of both.

In embodiments, the invention includes pharmaceutical formulations comprising sirolimus wherein the formulations, which are monolithic or multiparticulate, may be in matrix and/or reservoir form.

In an aspect, the invention relates to pharmaceutical formulations comprising sirolimus, wherein embodiments comprise:

a) an inert core;

b) a drug layer comprising sirolimus and at least one surface modifying agent, surrounding the core;

c) optionally, a barrier layer over the drug coated core; and

d) a sugar coating.

In embodiments, the invention includes pharmaceutical formulations comprising an inert core and a sugar overcoat, wherein the sugar overcoat comprises: (a) sirolimus or derivatives thereof; (b) at least one surface modifying agent; and (c) one or more sugar in an amount less than about 35% of the weight of the sugar overcoat.

In embodiments, the invention includes pharmaceutical formulations comprising: (a) an inert core; (b) a sugar overcoat comprising: (i) sirolimus or derivatives thereof; (ii) at least one surface modifying agent; and (iii) one or more sugar in an amount less than about 35% of the weight of the sugar overcoat; (c) optionally, an inert barrier layer; and (d) a smooth coating layer comprising at least mannitol.

In an aspect, the invention includes pharmaceutical formulations comprising sirolimus, wherein embodiments comprises:

a) an inert core;

b) a drug layer comprising sirolimus, at least one surface modifying agent, and a sugar surrounding the core, wherein the sugar is present in an amount less than about 35% of the weight of the layer;

c) optionally, an inert barrier layer applied onto the drug coated core; and

d) a sugar coating.

The inert cores are pharmacologically inert in nature and pharmaceutically compatible. An inert core may be a placebo tablet, prepared by compressing a mixture of excipients. Non-limiting examples of various substances that can be used for inert cores include insoluble inert materials such as glass particles/beads or silicon dioxide, calcium phosphate dihydrate, dicalcium phosphate, calcium sulfate dihydrate, microcrystalline cellulose, cellulose derivatives, calcium carbonate, dibasic calcium phosphate anhydrous, dibasic calcium phosphate monohydrate, tribasic calcium phosphate, magnesium carbonate, and magnesium oxide, soluble cores such as sugar spheres having sugars like dextrose, lactose, anhydrous lactose, spray-dried lactose, lactose monohydrate, mannitol, starches, sorbitol, and sucrose, insoluble inert plastic materials such as spherical or nearly spherical core beads of polyvinylchloride or polystyrene, and any other pharmaceutically acceptable insoluble synthetic materialic materials, and the like and mixtures thereof.

Sirolimus is poorly soluble in water. Increasing drug solubility and stability through appropriate formulation approaches can lead to increased therapeutic efficacy of the drug. Many approaches have been used to improve the solubility and dissolution properties of poorly soluble active ingredients including salt formation, particle size reduction, formation of nanoparticles, pH adjustment, use of surfactants, inclusion complexes, use of oily formulations, use of self-emulsifying drug delivery systems, formation of co-precipitates with hydrophilic polymers, co-milling with hydrophilic excipients, and others.

Being an insoluble drug, particle sizes of sirolimus could impact its solubility. The smaller the particle sizes, the greater the surface area; resulting in higher solubility and improved bioavailability.

As used herein, particle size is determined on the basis of the weight or volume average particle sizes as measured by particle size measuring techniques well known to those skilled in the art. Such techniques include, for example, sedimentation field flow fractionation, photon correlation spectroscopy, laser light scattering, such as using a Malvern particle size analyzer (Malvern Instruments Ltd., Malvern, Worcestershire, United Kingdom), and disk centrifugation.

In embodiments, the present invention relates to pharmaceutical formulations comprising sirolimus particles having effective average particle sizes greater than about 2 μm.

In embodiments, the present invention relates to pharmaceutical formulations comprising sirolimus particles having effective average particle sizes greater than about 400 nm, and less than about 5 μm, or less than about 3 μm, or less than about 2 μm, together with a surface modifying agent comprising poloxamer 188.

“Effective average particle size” as used herein refers to at least 50% of the drug particles having weight average particle sizes greater than about 2 μm, or greater than 400 nm but less than 2 μm.

Surfactants are wetting agents that lower the surface tension of a liquid, allowing easier spreading, and lower the interfacial tension between two liquids. They contain both hydrophobic groups and hydrophilic groups, thus being soluble in both organic solvents and water.

Surfactants may be ionic or nonionic. Ionic surfactants may be anionic, cationic, or zwitterionic. Anionic surfactants include the alkoyl isethionates, alkyl and alkyl ether sulfates and salts thereof, alkyl and alkyl ether phosphates and salts thereof, alkyl methyl taurates, and soaps, such as, for example, alkali metal salts including sodium or potassium salts of long chain fatty acids. Non-limiting examples include chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, and sodium lauryl sulfate (SLS), also called sodium dodecyl sulfate (SDS).

Examples of amphoteric and zwitterionic surfactants include but are not limited to carboxy, sulfonate, sulfate, phosphate, and phosphonate compounds. Examples are alkylimino acetates and iminodialkanoates and aminoalkanoates, imidazolinium and ammonium derivatives, betaines, sultaines, hydroxysultaines, alkyl sarcosinates and alkanoyl sarcosinates, and the like.

Nonionic surfactants include polyoxyethylene castor oil derivatives, polyoxyethylene sorbitan fatty acid esters (polysorbates, e.g., the commercially available Tween® products, including Tween 20 and Tween 800, from ICI Speciality Chemicals), poloxamers (e.g., Pluronic® products F68, F127 and F108Q, which are block copolymers of ethylene oxide and propylene oxide, from BASF Corporation), poloxamines (e.g., Tetronic® 908, also known as poloxamine 908, which is a tetrafunctional block copolymer derived from sequential addition of propylene oxide and ethylene oxide to ethylenediamine from BASF Wyandotte Corporation, Parsippany, N.J. USA), and Tetronic™ 15080 (T-1508) (BASF Wyandotte Corporation).

Examples of useful cationic surfactants include, but are not limited to, polymers, biopolymers, polysaccharides, cellulosics, alginates, phospholipids, and nonpolymeric compounds, such as zwitterionic stabilizers, poly-n-methylpyridinium, anthrylpyridinium chloride, cationic phospholipids, chitosan, polylysine, polyvinylimidazole, polybrene, polymethylmethacrylate, polyvinylpyrrolidone-2-dimethylaminoethyl methacrylate, lysozyme, long-chain polymers such as alginic acid, carrageenan (FMC Corp.), and POLYOX™ (Dow Chemical Co., Midland, Mich. USA), cationic lipids, sulfonium, phosphonium, and quarternary ammonium compounds, such as stearyltrimethylammonium chloride, and benzyl-di-(2-chloroethyl)ethylammonium bromide.

“Sugar” as used herein includes one or more of lactose, mannitol, sorbitol, starch, and sucrose.

In order to provide a barrier layer between “core and drug layering” or “drug layering and sugar coating” or “final film coating over sugar coating” or “coating over drug layer comprising sugar,” etc, various film-forming agents that can be used include, but are not limited to, cellulose derivatives such as soluble alkyl- or hydroalkyl-cellulose derivatives such as methylcelluloses, hydroxymethylcelluloses, hydroxyethylcelluloses, hydroxypropylcelluloses, hydroxymethyl ethylcelluloses, hydroxypropyl methylcelluloses, sodium carboxymethyl celluloses, etc., acidic cellulose derivatives such as cellulose acetate phthalates, cellulose acetate trimellitates, methylhydroxypropylcellulose phthalates, polyvinyl acetate phthalates, etc., insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acid, polyvinylalcohols, polyvinyl acetates, polyvinylpyrrolidones, polymethacrylates and derivatives thereof (e.g., Eudragit® products from Evonik Industries, Germany), and chitosan and derivatives thereof.

If desired, the films may contain additional adjuvants for the coating process such as plasticizers, polishing agents, colorants, pigments, antifoam agents, opacifiers, antisticking agents, and the like.

Various useful plasticizers include, but are not limited to, substances such as castor oil, diacetylated monoglycerides, dibutyl sebacate, diethyl phthalate, glycerin, polyethylene glycol, propylene glycol, triacetin; triethyl citrate. Also, mixtures of plasticizers may be utilized. The type of plasticizer depends upon the type of coating agent. A plasticizer is frequently present in amounts ranging from about 5% (w/w) to 30% (w/w) based on the total weight of the film coating.

An opacifier such as titanium dioxide may also be present in an amount ranging from about 10% (w/w) to about 20% (w/w), based on the total weight of the coating. When colored tablets are desired, the color is normally applied in the coating. Consequently, coloring agents and pigments may be present in the film coating. Various coloring agents include, but are not limited to, iron oxides, which can be red, yellow, black, or blends thereof.

Anti-adhesives are frequently used in film coating processes to avoid sticking effects during film formation and drying. An example of a useful anti-adhesive for this purpose is talc. The anti-adhesive can present in the film coating in amounts of about 5% (w/w) to 15% (w/w), based upon the total weight of the coating.

Suitable polishing agents include polyethylene glycols of various molecular weights or mixtures thereof, talc, surfactants (e.g. glycerol monostearate and poloxamers), fatty alcohols (e.g., stearyl alcohol, cetyl alcohol, lauryl alcohol and myristyl alcohol) and waxes (e.g., carnauba wax, candelilla wax and white wax). In embodiments, polyethylene glycols having molecular weights about 3,000-20,000 are employed.

In addition to above coating ingredients, sometimes pre-formulated coating products such as Opadry® (from Colorcon) are employed, requiring only dispersion in a liquid before use. Opadry compositions generally comprise material, plasticizer and, if desired, pigment in a dry concentrate, requiring only dispersion in a liquid before use. Opadry formulas produce attractive, elegant coatings on a variety of tablet cores and can be used in both aqueous and organic coating procedures.

Various solvents used in processes for preparing pharmaceutical formulations include, but are not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and mixtures thereof.

In an aspect, the invention includes processes for preparing pharmaceutical formulations of sirolimus, embodiments comprising:

a) dissolving or dispersing sirolimus in a suitable solvent together with one or more surface modifying agents;

b) optionally, adding sugar or a binder;

c) layering or applying the solution or dispersion from a) or b) onto inert cores;

d) optionally, applying a barrier coating;

e) applying a sugar coating; and

f) optionally, applying a film coating.

The coating steps comprise drug layering techniques or coating techniques known to the skilled artisan. For example, the drug layering or coating techniques may include, but are not limited to, powder coating, spray coating, dip coating, fluidized bed coating with Wurster or top spray or side spray techniques, and modifications thereof.

In embodiments the invention includes powder compositions comprising sirolimus and at least one pharmaceutically acceptable polymer.

Various pharmaceutically acceptable polymers that can be used in the context of the invention include, but are not limited to, polyethylene glycols (molecular weight≦about 400), hydroxymethyl celluloses, hydroxyethyl celluloses, hydroxypropyl celluloses, hydroxypropyl methylcelluloses (hypromelloses or HPMC), methyl celluloses, carboxymethyl celluloses (CMC), sodium CMC, carboxyethyl celluloses, carboxypolymethylene, hydroxypropylmethyl phthalate, polyvinylpyrrolidones, cellulose acetates, sodium alginate, gums such as acacia gum, guar gum, tragacanth gum and xanthan gum; methacrylic acid copolymers like poly(butylmethacrylate, (2-dimethylaminoethyl) methacrylate, methylmethacrylate, Eudragit® products designated E 100 or E 12.5 or E PO, polyvinyl acetal diethylaminoacetate (available as AEA supplied by Sankyo Co. Limited), chitosan, and the like and mixtures thereof.

In embodiments, the invention includes powder compositions comprising sirolimus, wherein sirolimus is in solubility enhanced form.

Some useful techniques for the preparation of powder compositions of sirolimus include, without limitation thereto, solvent evaporation, spray-drying, agitated thin-film drying, spray freezing, spray congealing, supercritical fluid precipitation, and other techniques known in the art. In certain embodiments, a solid dispersion intimate mixture (or solid solution) is formed by removing solvent from a solution comprising sirolimus and at least one pharmaceutically acceptable polymer.

Frequently it has been observed that during preparation of pre-mix powder compositions the active is in substantially amorphous form. But surprisingly it has been observed that sirolimus retains its crystallinity substantially in the powder compositions. This property of sirolimus may contribute to its stability in the compositions and/or formulations. This may be shown through the X-ray powder diffraction (XRD) patterns, wherein powder compositions retain the crystalline peaks attributed to sirolimus.

The composition or powder compositions may further comprise an antioxidant, to protect the drug from oxidative degradation. Antioxidants that are useful include vitamin E and derivatives thereof, ascorbic acid, sodium pyrosulphite, glutathion and sorbic acid.

Further embodiments of the invention include stable powder compositions comprising sirolumus.

The invention also relates to stable pharmaceutical compositions comprising sirolimus, and their respective formulations.

In embodiments of the present invention, the formulation as a whole, or at least partially, comprises compositions or powder compositions of sirolimus and at least one other pharmaceutically acceptable excipient such as binders, diluents, lubricant/glidants, disintegrating agents, surfactants, solvents, and coloring agents.

Non-limiting examples of binders include one or more of gum acacia, cholesterol, tragacanth, stearic acid, gelatin, casein, lecithin (phosphatides), carboxymethylcellulose calcium, carboxymethylcellulose sodium, methylcelluloses, hydroxyethylcellulose, hydroxypropylcellulose, hydroxypropyl methycellulose phthalates, microcrystalline celluloses, noncrystalline celluloses, polyvinylpyrrolidones (povidones or PVP), cetostearyl alcohol, cetyl alcohol, cetyl esters wax, dextrates, dextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, polyvinylalcohols, and mixtures thereof.

Non-limiting examples of diluents include calcium carbonate, calcium phosphate dibasic, calcium phosphate tribasic, calcium sulfate, microcrystalline cellulose, powdered cellulose, dextrates, dextrins, dextrose excipients, fructose, kaolin, lactitol, lactose, mannitol, sorbitol, starches, sucrose, and mixtures thereof.

Non-limiting examples of lubricants/glidants include colloidal silicon dioxide, stearic acid, magnesium stearate, calcium stearate, talc, hydrogenated castor oil, and mixtures thereof.

Non-limiting examples of disintegrants include starches, modified starches, croscarmellose sodium, crospovidones, and sodium starch glycolate.

Useful coloring agents include FDA approved colorants and examples are iron oxides, lake of tartrazine, allura red, lake of quinoline yellow, and lake of erythrosine.

In embodiments, the present invention relates to processes for preparation of pharmaceutical compositions and/or formulations.

In embodiments, the present invention relates to methods of using pharmaceutical formulations of the present invention, such as for the prophylaxis of organ rejection in patients age 13 years or older, receiving renal transplants.

Embodiments of methods of using pharmaceutical formulations of the present invention may include co-administration of cyclosporine and/or corticosteroids, as required for particular patients.

Formulations prepared as above can be subjected to in vitro dissolution evaluations according to Test 711 “Dissolution” in United States Pharmacopoeia 29, United States Pharmacopeial Convention, Inc., Rockville, Md., 2005 (“USP”), to determine the rate at which the active ingredient is released from the dosage forms, and content of active ingredient can conveniently be determined in solutions by techniques such as high performance liquid chromatography.

The formulations prepared can be packaged using appropriate packaging materials such as containers and closures composed of polyethylene (high density polyethylene or low density polyethylene), polypropylene, glass, stainless steel, etc. Also useful are various blisters or strips composed of aluminum or high-density polypropylene, or polyvinyl chloride, or polyvinyl chloride (PVC) coated with polyvinylidene dichloride (PVDC), generally termed PVC/PVDC. Different grades of PVC/PVDC are available as PVC/PVDC 40 gsm, PVC/PVDC 60 gsm, PVC/PVDC 90 gsm, etc., where “gsm” indicates the grams of PVDC coating per square meter of PVC film. As sirolimus is sensitive to the presence of oxygen and moisture, various moisture adsorbents like silica gel, molecular sieves, etc. and oxygen absorbers such as Ageless® (manufactured by Mitsubishi Gas Chemical), or Stabilox® (manufactured by Multisorb Technologies) can be included in packaged sirolimus products.

The following examples illustrate certain specific aspects and embodiments of the invention and demonstrate the practice and advantages thereof. It is to be understood that the examples are given by way of illustration only and are not intended to limit the scope of the invention in any manner.

### EXAMPLE 1

### Pharmaceutical Formulation Comprising Sirolimus 2 Mg

| | |
|---|---|
| Ingredient | mg/Tablet |

Core Tablets

| | |
|---|---|
| Lactose | 187 |
| Colloidal silicon dioxide (Aerosil | 2 |
| 200) | |
| Magnesium stearate | 1.5 |

Barrier Coating 1

| | |
|---|---|
| Pharmaceutical glaze$ | 10 |
| Calcium sulfate | 5 |
| Glyceryl monooleate | 1 |
| Water‡ | q.s. |

Drug Layering

| | |
|---|---|
| Sirolimus | 2 |
| Poloxamer 188 | 2 |
| Hydroxypropyl methylcellulose | 5 |
| (HPMC 5 cps) | |
| Microcrystalline cellulose | 0.1 |
| D,L-Tocopherol | 0.25 |
| Water‡ | q.s. |

Barrier Coating 2

| | |
|---|---|
| Hydroxypropyl methylcellulose | 10 |
| (HPMC 5 cps) | |
| Polyethylene glycol (PEG 400) | 1 |
| Water‡ | q.s. |

Sugar Coating

| | |
|---|---|
| Sucrose | 29.1 |
| Water‡ | q.s. |

Film Coating

| | |
|---|---|
| Oplalux# | 10 |

$Pharmaceutical glaze contains shellac and is supplied by Temuss.
#Opalux contains sugar, titanium dioxide, HPMC and colour, and is supplied by Colorcon.
‡Evaporates during processing

Manufacturing process:

A. Preparation of core tablets:

1) Lactose, colloidal silican dioxide, and magnesium stearate are sifted through an ASTM #40 mesh sieve.

2) The blend is compressed into tablets.

B. Barrier coating 1:

1) Pharmaceutical glaze is dissolved in water at 65-70° C. and allowed to cool to about 40° C.

2) Calcium sulfate and glycerol monoleate are added to step 1) to form a dispersion.

3) The core tablets from above are coated with the dispersion prepared in step 2).

C. Drug dispersion and layering:

1) Poloxamer 188 is dissolved in half the quantity of water and stirred to form a clear solution.

2) Sirolimus is added to the solution and stirred to form a uniform dispersion.

3) The dispersion is homogenised to achieve the desired particle sizes.

4) D,L-Tocopherol, microcrystalline cellulose, and HPMC are dispersed in the remaining half of water to form a uniform dispersion.

5) The dispersion of step 4) is added to drug dispersion of step 3) and stirred to form a homogenous dispersion.

6) The dispersion prepared in step 5) is layered onto barrier coated tablets prepared above.

D. Barrier coating 2:

1) Water is heated to about 65-70° C., and HPMC and PEG 400 are dissolved.

2) The drug layered tablets are coated with the solution.

E. Sugar coating:

1) Water is heated to about 65-70° C., sucrose is dissolved and the solution is cooled to about 40° C.

2) The tablets having barrier coating 2 prepared above are coated with the solution.

F. Film coating:

1) The sugar coated tablets prepared above are further coated with Opalux dispersion.

### EXAMPLE 2

### Pharmaceutical Formulation Comprising Sirolimus 2 Mg

| | |
|---|---|
| Ingredient | mg/Tablet |

Core Tablets

| | |
|---|---|
| Lactose (DCL 11) | 160 |
| Microcrystalline cellulose | 13 |
| (PH200) | |
| Polyethylene glycol (PEG 8000) | 25 |
| Magnesium stearate | 2 |

Seal Coating

| | |
|---|---|
| Opaglos NA 7150# | 10 |
| Ethanol‡ | 20 |

Barrier Coating 1

| | |
|---|---|
| Sucrose | 19 |
| Microcrystalline cellulose | 2 |
| (PH105) | |
| Hydroxypropyl methylcellulose | 2 |
| (HPMC 5 cps) | |
| Povidone K 30 | 2 |
| Water‡ | 60 |

Drug Layering

| | |
|---|---|
| Sirolimus | 2 |
| HPMC 5 cps | 5 |
| Poloxamer 188 | 1 |
| Microcrystalline cellulose | 0.1 |
| (PH105) | |
| HPMC 5 cps | 5 |
| D,L-Tocopherol | 0.1 |
| Ethanol‡ | 0.5 |
| Sucrose | 5 |
| Water‡ | 297 |

Barrier Coating 2

| | |
|---|---|
| Sucrose | 78.34 |
| Water | 60 |

Film Coating

| | |
|---|---|
| Opadry Yellow* | 12 |

‡Evaporates during processing.
#Opaglos NA 7150 contains shellac, povidone, methylated spirit, and PEG 8000, and is supplied by Colorcon.
*Opadry Yellow contains hydroxypropyl methylcellulose, titanium dioxide, macrogol, talc, and iron oxide yellow, and is supplied by Colorcon.

Manufacturing process: similar to that for Example 1.

The tablets of Example 2 and RAPAMUNE 2 mg tablets are subjected to dissolution testing in 500 mL of 0.4% sodium lauryl sulphate (SLS) in water, 120 RPM stirring, using USP I (basket 20 mesh) apparatus. The cumulative percentages of contained drug that dissolved are shown in Table 1.

TABLE 1

| Time (minutes) | RAPAMUNE ® 2 mg | Example 2 |
|---|---|---|
| 10 | 98 | 99 |
| 20 | 102 | 101 |
| 30 | 102 | 101 |
| 45 | 102 | 101 |
| 60 | 102 | 102 |
| 120 | 102 | 102 |

### EXAMPLES 3-4

Pharmaceutical Formulations Comprising Sirolimus 2 Mg.

mg/Tablet

| | | |
|---|---|---|
| Ingredient | Example 3 | Example 4 |

Core Tablets

| | | |
|---|---|---|
| Lactose (DCL 11) | 124 | 124 |
| Microcrystalline cellulose (PH200) | 32 | 32 |
| Polyethylene glycol 8000 | 22 | 22 |
| Magnesium stearate | 2 | 2 |

Seal Coating

| | | |
|---|---|---|
| Opaglos NA 7150 | 10 | 10 |
| Ethanol‡ | 20 | 20 |

Barrier Coating 1

| | | |
|---|---|---|
| Sucrose | 10 | 10 |
| Microcrystalline cellulose (PH105) | 1 | 1 |
| Hydroxypropyl methylcellulose | 2 | 2 |
| (HPMC 5 cps) | | |
| Water‡ | 48.5 | 50 |

Drug Layer

| | | |
|---|---|---|
| Sirolimus | 2 | 2 |
| Polysorbate 80 | 0.25 | 0.25 |
| Hydroxypropyl methylcellulose | 0.25 | 0.25 |
| (HPMC 5 cps) | | |
| Polyvinylpyrrolidone (PVP-K30) | — | 0.75 |
| Microcrystalline cellulose (PH105) | 0.25 | 0.25 |
| Hydroxypropyl methylcellulose | 2.25 | 2.25 |
| (HPMC 5 cps) | | |
| D,L-Tocopherol | 0.1 | 0.1 |
| Ethanol‡ | 0.5 | 0.5 |
| Sucrose | 25 | 48.4 |
| Water‡ | 164 | 78.25 |

Barrier Coating 2

| | | |
|---|---|---|
| Sucrose | 91.9 | 50 |
| Water‡ | q.s. | q.s. |

Film Coating

| | | |
|---|---|---|
| Hydroxypropyl methylcellulose | 8.9 | 8.9 |
| (HPMC 6 cps) | | |
| Titanium dioxide | 2.97 | 2.97 |
| Polyethylene glycol 20,000 | 0.75 | 0.75 |
| Iron oxide yellow | 0.72 | 0.72 |
| Iron oxide red | 0.06 | 0.06 |
| Sucrose | 1.5 | 1.5 |
| Water‡ | q.s. | q.s. |

‡Evaporates during processing.

Manufacturing process:

A. Core tablets:

1) Lactose, microcrystalline cellulose, PEG 8000 and magnesium stearate are sifted through an ASTM #40 mesh sieve and blended.

2) The blend from step 1) is compressed into tablets.

B. Seal coating:

1) Opagloss NA 7150 is dispersed in ethanol.

2) The core tablets prepared above are coated with the dispersion prepared in step 1).

C. Barrier coating 1:

1) Sucrose and HPMC are dissolved in water.

2) Microcrystalline cellulose is added to the solution.

3) The dispersion of 2) is stirred for 15-20 minutes, and then is used to coat the seal coated tablets prepared above.

D. Drug dispersion and layering:

1) Polysorbate 80 is dissolved in half of the water and stirred to form a clear solution.

2) HPMC 5 cps and sirolimus are added to the solution and stirred to form a uniform dispersion.

3) The dispersion is homogenized to achieve a particle size distribution with an effective average particle size of 2.1 μm.

4) D,L-Tocopherol, microcrystalline cellulose (PH105), and HPMC are dispersed in the other half of the water to form a uniform dispersion.

5) The dispersion prepared in step 4) is added to drug dispersion prepared of step 3) and stirred to form a homogenous dispersion.

6) The dispersion prepared in step 5) is layered onto the barrier coated tablets prepared above.

E. Barrier coating 2:

1) Water is heated to about 65-70° C., then sucrose is dissolved and the solution is cooled to about 40° C.

2) Coat the drug layered tablets prepared above with the sucrose solution.

F. Film coating and polishing:

1) HPMC 6 cps, sucrose and PEG 20000 are dissolved in water.

2) Titanium dioxide, iron oxide yellow and iron oxide red are dispersed in the solution and stirred for 45 minutes.

3) The tablets having barrier coating 2 prepared above are further coated with the dispersion.

The tablets of Examples 3 and 4, and RAPAMUNE 2 mg tablets, are subjected to dissolution testing, as described in Example 2. The cumulative percentages of drug dissolved for each sample are shown in Table 2.

TABLE 2

| Time (minutes) | RAPAMUNE 2 mg | Example 3 | Example 4 |
|---|---|---|---|
| 10 | 98 | 96 | 90 |
| 20 | 102 | 98 | 96 |
| 30 | 102 | 98 | 99 |
| 45 | 102 | 98 | 100 |
| 60 | 102 | 98 | 100 |
| 120 | 102 | 98 | 101 |

### EXAMPLES 5-6

### Pharmaceutical Formulations Comprising Sirolimus 2 Mg

mg/Tablet

| | | |
|---|---|---|
| Ingredient | Example 5 | Example 6 |

Core Tablets

| | | |
|---|---|---|
| Lactose | 187 | 187 |
| Colloidal silicon dioxide | 2 | 2 |
| Magnesium stearate | 1.5 | 1.5 |

Barrier Coating 1

| | | |
|---|---|---|
| Pharmaceutical glaze | 10 | 10 |
| Calcium sulfate | 5 | 5 |
| Glyceryl monooleate | 1 | 1 |
| Water‡ | q.s. | q.s. |

Drug Layer

| | | |
|---|---|---|
| Sirolimus | 2 | 2 |
| Poloxamer 407 | 2 | 2 |
| Hydroxypropyl methylcellulose | 5 | 5 |
| (HPMC 5 cps) | | |
| Sucrose | — | 10 |
| Microcrystalline cellulose | 0.1 | 0.1 |
| D,L-Tocopherol | 0.25 | 0.25 |
| Water‡ | q.s. | q.s. |

Barrier Coating 2

| | | |
|---|---|---|
| Hydroxypropyl methylcellulose | 10 | 10 |
| (HPMC 5 cps) | | |
| Polyethylene glycol 400 | 1 | 1 |
| Water‡ | q.s. | q.s. |

Sugar Coating

| | | |
|---|---|---|
| Sucrose | 29.1 | 29.1 |
| Water‡ | q.s. | q.s. |

Film Coating

| | | |
|---|---|---|
| Oplalux | 10 | 10 |

‡Evaporates during processing.

Manufacturing process: similar to that of Example 1.

### EXAMPLES 7-9

### Formulations Comprising Sirolimus

| | mg/Tablet | | |
|---|---|---|---|
| Ingredient | Example 7 | Example 8 | Example 9 |
| Sirolimus | 2 | 2 | 2 |
| Povidone (PVP K 30) | 1 | 1 | — |
| Polysorbate 80 | 1 | — | — |
| Poloxamer 188 | — | 1 | — |
| Poloxamer 407 | — | — | 1 |
| D,L-Tocopherol | 0.1 | 0.1 | 0.1 |
| Methanol‡ | q.s. | q.s. | q.s. |
| Sucrose | 110 | 110 | 110 |

Extragranular

| | | | |
|---|---|---|---|
| Sucrose (DC grade) | 37.9 | 37.9 | 100 |
| Microcrystalline cellulose | 40 | 40 | — |
| PH102 | | | |
| Microcrystalline cellulose | — | — | 50 |
| PH200 | | | |
| Croscarmellose sodium | 10 | 10 | — |
| Hydrogenated castor oil | 8 | 8 | 8 |
| Magnesium stearate | 0.05 | 0.05 | — |
| Sodium stearyl fumarate | — | — | 0.2 |

Smooth Coating

| | | | |
|---|---|---|---|
| Sucrose | 29.05 | 29.05 | 54 |
| Water‡ | q.s. | q.s. | q.s. |

Colour Coating

| | | | |
|---|---|---|---|
| Opadry Yellow | 10 | 10 | — |
| Hydroxypropyl methylcellulose | — | — | 8.7 |
| (HPMC 6 cps) | | | |
| Titanium dioxide | — | — | 2.97 |
| PEG 20000 | — | — | 0.75 |
| Iron oxide yellow | — | — | 0.72 |
| Iron oxide red | — | — | 0.06 |
| Sucrose | 0.04 | 0.04 | 1.45 |
| Water‡ | q.s. | q.s. | q.s. |

Polishing

| | | | |
|---|---|---|---|
| Carnauba wax | 0.05 | 0.05 | 0.05 |

‡Evaporates during processing.

Manufacturing process:

A. Drug premix:

1) Sirolimus, D,L-tocopherol, and polaxamer 188, polysorbate 80, or poloxamer 407 are dissolved in methanol.

2) Sucrose is added to the solution of step 1.

3) The mixture of step 2 is evaporated using a Buchi Rotavapor, and then sifted through a sieve.

B. Blending, lubrication and compression:

1) Sucrose and microcrystalline cellulose are sifted through an ASTM #40 mesh sieve and blended with the drug premix prepared above.

2) Hydrogenated castor oil and sodium stearyl fumarate are passed through an ASTM #60 mesh sieve and added to materials of step 1).

3) The blend from step 2) is compressed into tablets.

C. Smooth coating:

1) Sucrose is dissolved in water at 65-70° C. and the solution is cooled to about 40° C.

3) The tablets prepared above are coated with the solution of step 1).

D. Colour coating:

1) Opalux is dispersed in water to form a coating material.

2) The smooth coated tablets prepared above are coated with the dispersion prepared in step 1).

E. Polishing:

1) The colour coated tablets prepared above are polished with carnauba wax.

The tablets of Examples 8 and 9 are packaged in closed HDPE containers and stored under accelerated stability testing conditions of 40° C. and 75% RH for 1 month, and the formulations are analyzed for physical stability by XRD. The drug is found to be in crystalline form for both of the formulations, both before and after the storage.

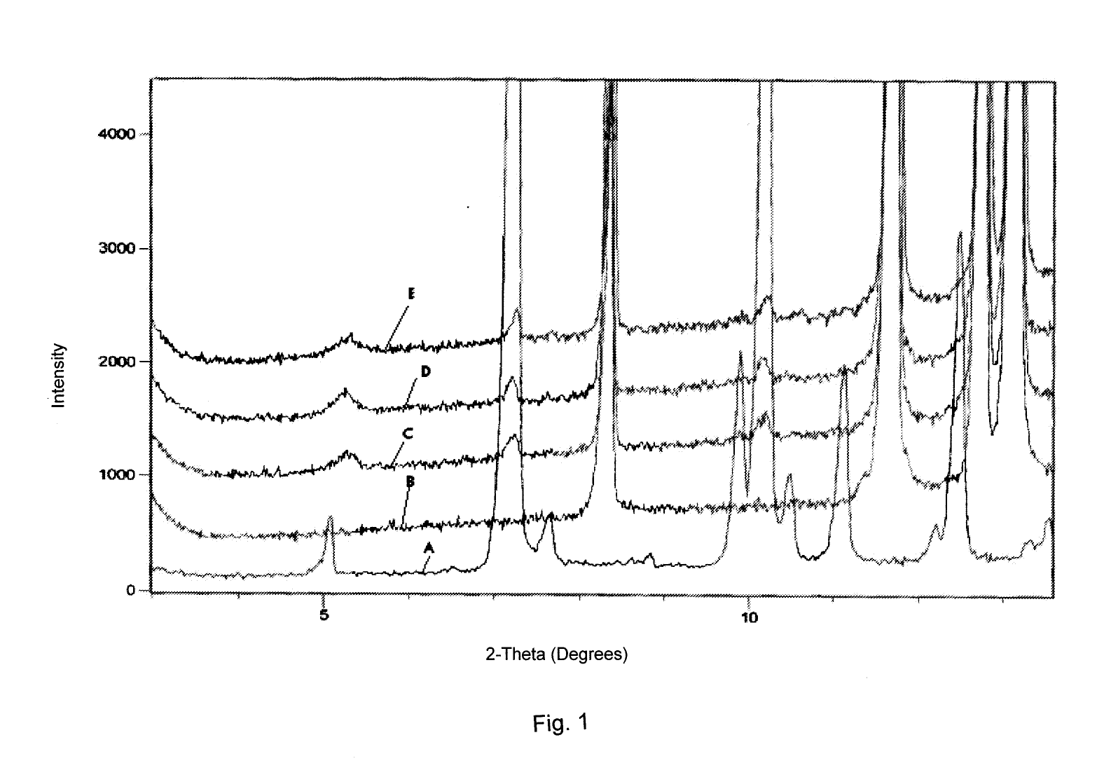

XRD patterns for Example 8 materials are shown in FIG. 1, where “A” is the pattern for crystalline sirolimus, “B” is the pattern for a composition prepared according to Example 8, but omitting the sirolimus, “C” is a pattern for a composition prepared according to Example 8, “D” is a pattern for a composition of Example 8 after storage at 40° C. and 75% RH for one month, and “E” is a pattern for a composition of Example 8 after storage at 40° C. and 75% RH for two months.

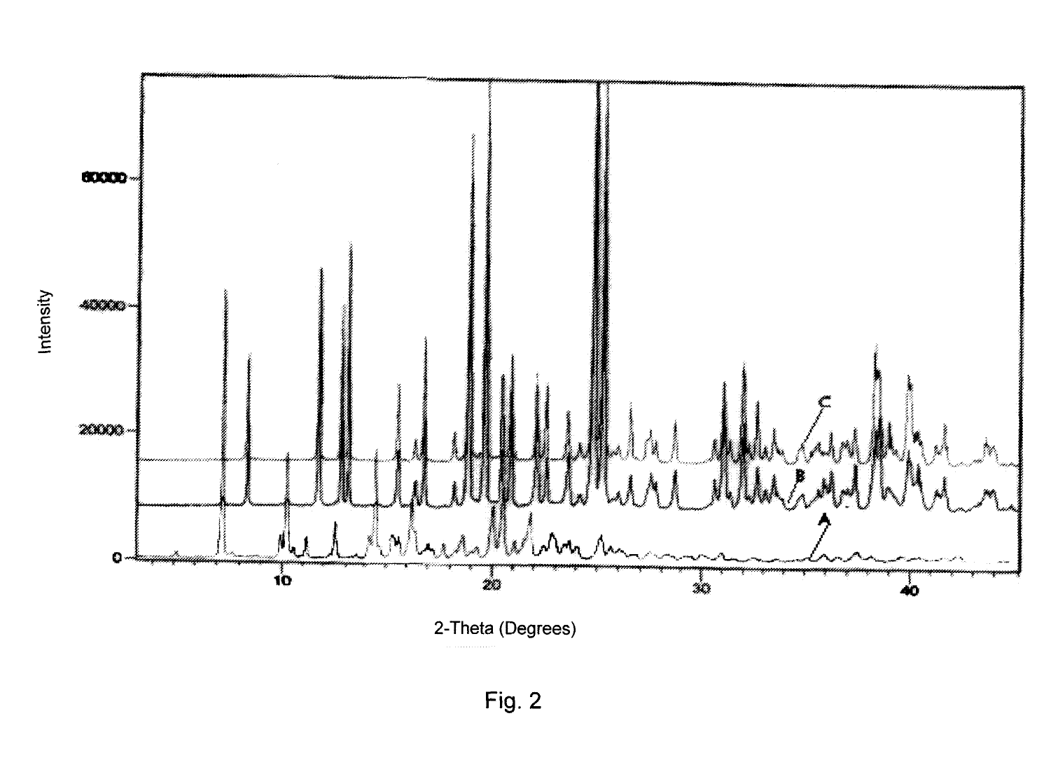

FIG. 2 shows XRD patterns of a crystalline sirolimus (“A”), a composition prepared according to Example 9, but without sirolimus (“B”), and a composition prepared according to Example 9 (“C”).

### EXAMPLES 10-11

### Formulations Comprising Sirolimus

mg/Tablet

| Ingredient | Example 10 | Example 11 |
|---|---|---|
| Sirolimus | 2 | 2 |
| Microcrystalline cellulose | 2 | 2 |
| Sucrose | 94.8 | 94.8 |
| Poloxamer 188 | 1 | — |
| Polysorbate 80 | — | 1 |
| D,L-Tocopherol | 0.1 | 0.1 |
| Methanol‡ | q.s. | q.s. |
| Hydrogenated castor oil | 4 | 4 |
| Sodium stearyl fumarate | 0.1 | 0.1 |

Smooth Coating

| | | |
|---|---|---|
| Sucrose | 30 | 30 |
| Water‡ | q.s. | q.s. |

Color Coating

| | | |
|---|---|---|
| Opalux | 2.9 | 2.9 |

‡Evaporates during processing.

Manufacturing process:

1) Sirolimus, polaxamer 188 or polysorbate 80, microcrystalline cellulose, most of the sucrose and a part of hydrogenated castor oil are sifted through an ASTM #40 mesh sieve.

2) D, L-Tocopherol is dissolved in methanol, the remaining quantity of sucrose is added, and the mixture is air dried.

3) The materials of steps 1) and 2) are blended.

4) Hydrogenated castor oil (remaining part) and sodium stearyl fumarate are passed through an ASTM #60 mesh sieve and are blended with materials of step 3).

5) The blend from step 4 is compressed into tablets.

6) The tablets are coated with sucrose solution.

7) The tablets prepared above are further coated with Opalux dispersion.

### EXAMPLE 12

### Formulation Comprising Sirolimus 2 Mg

| | |
|---|---|
| Ingredient | mg/Tablet |

Core Tablet

| | |
|---|---|
| Lactose (DCL 11) | 126 |
| Microcrystalline cellulose (Avicel PH200) | 32 |
| Polyethylene glycol 8000 | 20 |
| Magnesium stearate | 2 |

Seal Coating

| | |
|---|---|
| Opaglos NA 7150 | 10 |
| Isopropyl alcohol‡ | q.s. |

Subcoating

| | |
|---|---|
| Mannitol | 10 |
| Microcrystalline cellulose (Avicel PH105) | 1 |
| Hypromellose (5 cps) | 2 |
| Water‡ | q.s |

Drug Layering (Part 1)

| | |
|---|---|
| Sirolimus | 1 |
| Poloxamer 188 | 0.5 |
| Sucrose | 2.5 |
| Hypromellose (5 cps) | 3.5 |
| Microcrystalline cellulose (Avicel PH105) | 0.125 |
| Vitamin E | 0.05 |
| Ethanol‡ | 0.25 |
| Water‡ | q.s. |

Subcoating

| | |
|---|---|
| Mannitol | 10 |
| Hypromellose (5 cps) | 2 |
| Microcrystalline cellulose (Avicel PH105) | 1 |
| Water‡ | q.s. |

Barrier Coating layer 1

| | |
|---|---|
| Ethylcellulose (10 cps) | 1.15 |
| HPMC 5 cps | 3.05 |
| PEG 20000 | 0.3 |
| Isopropyl alcohol‡ | q.s. |
| Dichloromethane‡ | q.s. |

Subcoating

| | |
|---|---|
| Mannitol | 10 |
| Hypromellose (5 cps) | 2 |
| Microcrystalline cellulose (Avicel PH105) | 1 |
| Water‡ | q.s. |

Drug Layering (Part 2)

| | |
|---|---|
| Sirolimus | 1 |
| Poloxamer 188 | 0.5 |
| Sucrose | 2.5 |
| Hypromellose (5 cps) | 3.5 |
| Microcrystalline cellulose (Avicel PH105) | 0.125 |
| Vitamin E | 0.05 |
| Ethanol‡ | 0.25 |
| Water‡ | q.s. |

Smooth Coating

| | |
|---|---|
| Mannitol | 22 |
| Hypromellose (5 cps) | 4 |
| Water‡ | q.s. |

Film Coating

| | |
|---|---|
| Hypromellose (5 cps) | 6.95 |
| Titanium dioxide | 2.16 |
| PEG 20000 | 0.56 |
| Iron oxide yellow | 0.375 |
| Iron oxide red | 0.005 |
| Water‡ | q.s. |

Polishing

| | |
|---|---|
| Carnauba wax | 0.1 |

Imprinting ink

| | |
|---|---|
| Opacode red S-1-15052\hat{ }\hat{ } | q.s. |

‡Evaporates during processing.
\hat{ }\hat{ }Opacode Red S-1-15052 contains shellac, titanium dioxide, alcohol, FD&C Red 40 Allure Lake, n-butyl alcohol, propylene glycol, and ammonium hydroxide, and is supplied by Colorcon.

Manufacturing process:

1. Preparation of core tablets:

1.1. Lactose, microcrystalline cellulose, PEG 8000 and magnesium stearate are combined, sifted through an ASTM 40 mesh sieve, and blended for 10 minutes.

1.2. The blend from step 1.1 is compressed into tablets.

2. Seal coating:

2.1. Mix Opaglos NA 7150 and isopropyl alcohol.

2.2. Core tablets from step 1.2 are coated with the dispersion prepared in step 2.1.

3. Subcoating:

3.1. Dissolve mannitol and HPMC in water.

3.2. Disperse microcrystalline cellulose in the solution prepared in step 3.1

3.3. Coat the tablets prepared in step 2.2.

4. Preparation of drug dispersion and layering:

4.1. Poloxamer 188 is dissolved in warm water.

4.2. HPMC 5 cps and sirolimus are added to the solution prepared in step 4.1 and stirred to form a uniform dispersion.

4.3. The dispersion prepared in step 4.2 is homogenized to achieve a particle size distribution having 90% of the particles below 2-2.5 μm.

4.4. Vitamin E dissolved in ethanol, sucrose, MCC(PH105) and HPMC are dispersed in water to form a uniform dispersion.

4.5. The dispersion prepared in step 4.4 is added to drug dispersion prepared in step 4.3 and stirred to form a homogenous dispersion.

4.6. The dispersion of step 4.5 is divided into two equal parts.

4.7. Half of the dispersion is stirred for 15-20 minutes, and is used for coating of the tablets prepared in step 3.3.

5. Subcoating:

5.1. Dissolve mannitol and HPMC in water.

5.2. Disperse MCC(PH 105) in the solution prepared in step 5.1

5.3. The dispersion is stirred for 15-20 minutes, and used for subcoating of the tablets prepared in step 4.7.

6. Barrier coating layer 1:

6.1 Disperse ethylcellulose, HPMC 5 cps, and PEG 20,000 in isopropyl alcohol.

6.2. Add dichloromethane to the dispersion prepared in step 6.1.

6.3, Stir the dispersion for 15-20 minutes, and use to coat tablets prepared in step 5.3.

7. Subcoating:

7.1. Dissolve mannitol and HPMC in water.

7.2. Disperse MCC(PH 105) in the solution prepared in step 7.1.

7.3. The dispersion is stirred for 15-20 minutes, and is used to subcoat the tablets prepared in step 6.3.

8. Drug layering:

8.1. The second half of the drug dispersion of step 4.6 is coated onto the tablets prepared in step 7.3.

9. Smooth coating:

9.1. Dissolve mannitol and HPMC in water.

9.2. Disperse MCC(PH 105) in the solution prepared in step 9.1

9.3. The dispersion is stirred for 15-20 minutes, and is used to coat the tablets prepared in step 8.1.

10. Film coating and polishing:

10.1. HPMC 6 cps, sucrose and PEG 20000 are dispersed in water.

10.2. Titanium dioxide, iron oxide yellow, and iron oxide red are dispersed in the dispersion prepared in step 10.1 and stirred for 45 minutes.

10.3. The tablets prepared in step 9.3 are coated with the dispersion prepared in the step 10.2.

10.4. The tablets prepared in step 10.3 are polished with carnauba wax.

10.5. The tablets prepared in step 10.4 are imprinted with Opacode red S-1-15052.

We claim:

1. A pharmaceutical formulation, comprising a pharmacologically inert core having a coating comprising: (a) sirolimus or a derivative thereof; (b) at least one surface modifying agent; and (c) one or more sugars in an amount less than about 35% of the weight of the sugar overcoat.

2. The pharmaceutical formulation of claim 1, wherein a surface modifying agent comprises at least one of a polyoxyethylene castor oil derivative, a polyoxyethylene sorbitan fatty acid ester, a poloxamer, and a poloxamine.

3. The pharmaceutical formulation of claim 1, wherein a sugar comprises one or more of lactose, mannitol, sorbitol, a starch, and sucrose.

4. The pharmaceutical formulation of claim 1, wherein sirolimus used in preparation has an effective average particle size greater than about 400 nm and less than about 2 μm, and a surface modifying agent comprises poloxamer 188.

5. A method for the prophylaxis of organ rejection in patients receiving renal transplants comprising administering to a patient in need thereof a pharmaceutical formulation of claim 1.

6. The method of prophylaxis according to claim 5, comprising co-administering cyclosporine, a corticosteroid, or both.

7. A pharmaceutical formulation, comprising:
(a) a pharmacologically inert core;
(b) a coating over the core comprising: (i) sirolimus or a derivative thereof; (ii) at least one surface modifying agent; and (iii) one or more sugars in an amount less than about 35% of the weight of the coating;
(c) optionally, an inert barrier layer over the coating of (b); and
(d) a smooth coating layer comprising at least mannitol.

8. The pharmaceutical formulation of claim 7, wherein a surface modifying agent comprises at least one of a polyoxyethylene castor oil derivative, a polyoxyethylene sorbitan fatty acid ester, a poloxamer, and a poloxamine.

9. The pharmaceutical formulation of claim 7, wherein a sugar comprises one or more of lactose, mannitol, sorbitol, a starch, and sucrose.

10. The pharmaceutical formulation of claim 7, wherein an inert barrier layer comprises one or more of a methylcellulose, an ethylcellulose, a hydroxymethylcellulose, a hydroxyethylcellulose, a hydroxypropylcellulose, a hydroxymethyl ethylcellulose, a hydroxypropyl methylcellulose, a sodium carboxymethyl cellulose, a cellulose acetate phthalate, a cellulose acetate trimellitate, a methylhydroxypropylcellulose phthalate, a polyvinyl acetate phthalate, a dextrin, a starch, a starch derivative, a natural gum, gum Arabic, a xanthan, an alginate, a polyacrylic acid, a polyvinylalcohol, a polyvinyl acetate, a polyvinylpyrrolidone, a polymethacrylate or derivative thereof, and chitosan or a derivative thereof.

11. The pharmaceutical formulation of claim 7, wherein a smooth coating layer comprises mannitol and another pharmaceutically acceptable excipient.

12. The pharmaceutical formulation of claim 7, wherein sirolimus used in preparation has an effective average particle size greater than about 400 nm and less than about 2 μm, and a surface modifying agent comprises poloxamer 188.

13. A method for the prophylaxis of organ rejection in patients receiving renal transplants comprising administering to a patient in need thereof a pharmaceutical formulation of claim 7.

14. The method of prophylaxis of claim 13, comprising co-administering cyclosporine, a corticosteroid, or both.

15. A pharmaceutical formulation, comprising:
a) a pharmacologically inert core;
b) a coating over the core, comprising: (i) sirolimus or a derivative thereof; (ii) a surface modifying agent comprising a poloxamer or polysorbate; and (iii) one or more sugars in an amount less than about 35% of the weight of the coating; and
c) one or more polymer coatings, over the coating of b).

16. The pharmaceutical formulation of claim 15, wherein a surface modifying agent comprises poloxamer 188.

17. The pharmaceutical formulation of claim 15, further comprising a barrier coating between a) and b).

18. A pharmaceutical formulation comprising a tablet containing sirolimus and a surface modifying agent.

19. The pharmaceutical formulation of claim 18, wherein a surface modifying agent comprises a poloxamer.

20. The pharmaceutical formulation of claim 18, wherein a surface modifying agent comprises a polysorbate.
